(19) United States
(12) Patent Application Publication
(10) Pub. No.: US 2014/0276874 A1
(43) Pub. Date: Sep. 18, 2014

(54) ACETABULAR CUP IMPACTOR

(71) Applicant: In'Tech Medical, Inc., Rang-du-Fliers (FR)

(72) Inventors: Laurent PRUVOST, Neufchatel-Hardelot (FR); Xavier LEROY, Campigneulles-les-petites (FR)

(73) Assignee: In'Tech Medical, Inc., Rang-du-Fliers (FR)

(21) Appl. No.: 14/210,593

(22) Filed: Mar. 14, 2014

Related U.S. Application Data
(60) Provisional application No. 61/782,490, filed on Mar. 14, 2013.

(51) Int. Cl. A61F 2/46

(52) U.S. Cl. CPC A61F 2/4609; A61F 2002/30187
USPC 606/91

(57) ABSTRACT

An acetabular cup impactor for surgery. The impactor has a handle, a bow arm and a prehension fastener secured to the bow arm opposite the handle. An elongated spine extends within the length of the bow arm between the handle and the prehension fastener. In use, the elongated spine rotates with respect to the bow arm and causes corresponding rotation of the prehension fastener with respect to the bow arm.

### CROSS-REFERENCE TO RELATED APPLICATION

This application claims the priority benefit of U.S. Provisional Patent Application Ser. No. 61/782,490, filed Mar. 14, 2013, which is hereby incorporated herein by reference.

### TECHNICAL FIELD

The present invention relates generally to a medical device tool for performing hip surgery, and more specifically to an acetabular cup impactor.

### BACKGROUND

In hip surgery, an acetabular cup is placed within a cavity that houses an artificial femoral head. In order to gain access to the cavity to apply the acetabular cup, an elongated impactor is often used to secure, rotate and lock the cup into the cavity housing. Many existing instruments on the market, have a mechanism to put in place an acetabular cup. The general shape of those instruments has to respect the anatomy, and are usually curved. But, the challenge is to impart a rotation from the back of instrument (proximal end) to the acetabular cup (distal end), throughout a complex curved structure. The instruments already on the market are limited in angle and are difficult to clean. With the proposed design we solve this issue

Accordingly, it can be seen that there exists a need for a better way for installing an acetabular cup for hip surgery. It is to the provision of solutions to this and other problems that the present invention is primarily directed.

### SUMMARY

Generally described, the present invention relates to an acetabular cup impactor for surgery. The impactor has a handle, a bow arm and a prehension fastener secured to the bow arm opposite the handle. An elongated spine extends within the length of the bow arm between the handle and the prehension fastener. In use, the elongated spine rotates with respect to the bow arm and causes corresponding rotation of the prehension fastener with respect to the bow arm.

In a first example embodiment, the invention relates to an apparatus for surgery. The apparatus includes an elongated bow arm with a substantially arced shape. The elongated bow includes an elongated channel extending between a proximal end and a distal end. The apparatus also includes a handle that is rotatably secured with respect to the elongated bow arm proximal end. The apparatus also includes a spine that is received within the elongated bow arm channel and includes at least one rigid rod with distal ends and proximal ends and has a shape that substantially follows the substantially arced elongated bow arm. The spine is rotatably controlled by the handle.

In a second example embodiment, the invention relates to an apparatus for surgery. The apparatus includes a spine with at least one rigid rod. Each rigid rod includes a proximal section and a distal section separated by a horizontal axis. Each rigid rod also includes a linear incline section extending from the proximal section along an axis oblique to the horizontal axis. Each rigid rod further includes a linear decline section extending toward the distal section along an axis oblique to the horizontal axis. The apparatus also includes a rotatable handle rotatably secured with respect to the spine proximal end. Rotation of the rotatable handle causes a rotation of the spine.

In a third example embodiment, the invention relates to a method for performing surgery. The method includes removably securing medical insert to a prehension fastener. The prehension fastener is removably secured to the distal end of a spine that is received within a bow arm that has a distal end and a proximal end. The spine includes at least one rigid rod. Each rigid rod includes a proximal section and a distal section that are separated by a horizontal axis. Each rigid rod also includes a linear incline section that extends from the proximal section along an axis oblique to the horizontal axis. Each rigid rod further includes a linear decline section that extends toward the distal section along an axis oblique to the horizontal axis. The method further includes removably securing a rotatable handle to the proximal end of the bow arm. The method also includes controlling the rotation of the spine by rotating the rotatable handle. Rotating the handle causes equivalent rotation of the prehension fastener.

The specific techniques and structures employed to improve over the drawbacks of the prior devices and accomplish the advantages described herein will become apparent from the following detailed description of example embodiments and the appended drawings and claims.

### BRIEF DESCRIPTION OF THE DRAWINGS

FIG. 1 is a perspective view of an acetabular cup impactor according to a first example embodiment of the present invention.

FIG. 2 is a top view of the acetabular cup impactor of FIG. 1.

FIG. 3 is a side view of the acetabular cup impactor of FIG. 1.

FIG. 4 is a side view of the acetabular cup impactor of FIG. 1, showing the curvature of the bow arm with respect to an egg.

FIG. 5 is a perspective view of the acetabular cup impactor of FIG. 1, shown with the exterior partially transparent so as to view the internal components.

FIG. 6 is a side view of the acetabular cup impactor of FIG. 1, shown with the exterior partially transparent so as to view the internal components.

FIG. 7 is a perspective view of the acetabular cup impactor of FIG. 1 with the handle separated from the bow arm, shown with the exterior partially transparent so as to view the internal components.

FIG. 8 is an isolated side view of the bow arm of FIG. 1, shown with the exterior partially transparent so as to view the internal components.

FIG. 9 is a perspective section view of the connection between the bow arm and the handle of the acetabular cup impactor of FIG. 1, shown with the exterior partially transparent so as to view the internal components.

FIG. 10 is an isolated side view of the bow arm of FIG. 1 with the elongated spine pivoted out of the bow arm channel, shown with the exterior partially transparent so as to view the internal components.

FIG. 11 is a perspective section view of the proximal end of the bow arm of the acetabular cup impactor of FIG. 1.

FIG. 12 is an isolated top view of the bow arm of the acetabular cup impactor of FIG. 1.

FIG. 13 is an isolated perspective view of the elongated spine of FIG. 10.

FIG. 14 is a side view of one of the rods from the elongated spine of FIG. 13.

FIG. 15 is an isolated perspective view of the proximal end of the elongated spine of FIG. 13 removed from the distal head of the handle of FIG. 7.

FIG. 16 is an isolated perspective sectional view of the proximal end of the elongated skeleton of FIG. 13 inserted into the distal head of the handle of FIG. 7.

FIG. 17 is an isolated perspective view of the bow arm, tension cable and wrench of FIG. 1 with the elongated spine removed from the bow arm channel, shown with the exterior partially transparent so as to view the internal components and the wrench secured to the proximal end of the bow arm.

FIG. 18 is an isolated perspective view of the bow arm and wrench of FIG. 17, shown with the wrench removed from the proximal end of the bow arm.

FIG. 19 is an isolated rear perspective view of the bow arm and wrench of FIG. 1, shown with the wrench secured to the proximal end of the bow arm.

FIG. 20 is an isolated top perspective view of the bow arm and wrench of FIG. 1, shown with the wrench secured to the proximal end of the bow arm.

FIG. 21 is an isolated rear perspective view of the bow arm and wrench of FIG. 1, shown with the wrench secured to the proximal end of the bow arm, and shown with the wrench exterior partially transparent so as to view the internal components and the wrench secured to the proximal end of the bow arm.

FIG. 22 is an isolated top view of the bow arm and wrench of FIG. 1, shown with the wrench secured to the proximal end of the bow arm, and shown with the wrench exterior partially transparent so as to view the internal components and the wrench secured to the proximal end of the bow arm.

FIG. 23 is an isolated top view of the bow arm, wrench, translating ring and handle of FIG. 1, shown with the wrench and translating ring secured in a distal unlocked position to the proximal end of the bow arm, and shown with the wrench exterior partially transparent so as to view the internal components and the wrench secured to the proximal end of the bow arm.

FIG. 24 is an isolated top view of the bow arm, wrench, translating ring and handle of FIG. 1, shown with the wrench and translating ring secured in a transitional traction position to the proximal end of the bow arm, and shown with the wrench exterior partially transparent so as to view the internal components and the wrench secured to the proximal end of the bow arm.

FIG. 25 is an isolated top view of the bow arm, wrench, translating ring and handle of FIG. 1, shown with the wrench and translating ring secured in a proximal locked position to the proximal end of the bow arm, and shown with the wrench exterior partially transparent so as to view the internal components and the wrench secured to the proximal end of the bow arm.

FIG. 26 is an isolated perspective view of the handle of FIG. 1, shown with the knob attached.

FIG. 27 is an isolated perspective view of the handle of FIG. 1, shown with the knob detached.

FIG. 28 is an isolated interior view of the knob of FIG. 27 detached from the shaft that extends within the handle of FIG. 1.

FIG. 29 is an isolated rear perspective view of the proximal support of the handle of FIG. 1, and the shaft of FIG. 28 extending therethrough.

FIG. 30 is an isolated perspective view of the shaft of FIG. 28 removed from within the handle of FIG. 1.

FIG. 31 is an isolated perspective view of the shaft of FIG. 28 inserted through the handle of FIG. 1, shown without the knob of FIG. 27.

FIG. 32 is an isolated perspective view of the distal end of the bow arm of FIG. 1, shown with the exterior partially transparent so as to view the internal components such as the swivel receiver and the tension cable.

FIG. 33 is an isolated side perspective view of the swivel receiver and tension cable of FIG. 32, shown with a pair of fasteners according to a first example embodiment.

FIG. 34 is an isolated side perspective view of the swivel receiver and tension cable of FIG. 32, shown with a pair of fasteners according to a second example embodiment.

FIG. 35 is an isolated side perspective view of the swivel receiver and tension cable of FIG. 32, shown with a pair of fasteners according to a third example embodiment.

FIG. 36 is an isolated side sectional view of the translating ring within the wrench shown in FIG. 23, shown with the tension cable of FIG. 32 secured to the translating ring with a fastener.

FIG. 37 is a cross sectional view of the bow arm and tension cable of FIG. 17.

FIG. 38 is an isolated underneath perspective view of the distal end of the bow arm of FIG. 32, shown with the swivel receiver and tension cable removed.

FIG. 39 is a bottom perspective view of the bow arm of FIG. 1.

FIG. 40 is a perspective view of the elongated spine of FIG. 13 with the prehension fastener secured to the elongated spine distal end.

FIG. 41 is a perspective view of the elongated spine secured within the bow arm of FIG. 13, shown with the prehension fastener of FIG. 40 detached from the elongated spine, shown with the bow arm exterior partially transparent so as to view the internal components.

FIG. 42 is a perspective view of the elongated spine and prehension fastener of FIG. 40 secured to each other within the bow arm of FIG. 1 and a pipe collar and closing ring detached, shown with the bow arm exterior partially transparent so as to view the internal components.

### DETAILED DESCRIPTION OF EXAMPLE EMBODIMENTS

An example impactor 10 is depicted in FIGS. 1-42. The example impactor 10 has a handle portion 12, a bow arm 14 and a prehension fastener 190 secured with respect to the bow arm opposite the handle. An elongated spine 32 extends within the length of the bow arm 14 between the handle portion 12 and the prehension fastener 190. In use, the elongated spine 32 rotates within and with respect to the bow arm 14 and causes corresponding rotation of the prehension fastener 190 with respect to the bow arm. The handle portion 12 can be secured to the bow arm 14 with a fastener, for example corresponding threading, or through a more permanent connection, for example welding or adhesive. As depicted particularly in the top view of FIG. 2, when secured together, the handle portion 12 and bow arm 14 can extend along a single longitudinal axis R.

The example impactor 10 can be used to install multiple alternative example surgical products including, but not limited to, a cage inserter (TILF) an offset pedicle screw inserter, iliac or cervical screw, and an acetabular cup.

The handle portion 12 includes a rotatable handle or knob 18 at the proximal rear end and has a stopper 13 and neck 15 at the distal front end. As particularly depicted in FIGS. 5-7, the handle portion 12 has a grip 24. The handle portion 12 is generally rigid and durable to support the weight of the bow portion 14 when a user holds the impactor 10 by the grip 24. The grip 24 can include an overmold that is constructed of a durable and shock-absorbing material, for example silicone. The proximal end of the handle portion 12 includes a support 120 that can be disc-shaped with a circumferential groove 132 to receive an annular ring 134. The annular ring 134 can be constructed of a flexible material, for example plastic, or an elastic material. The annular ring 134 has a diameter similar to the internal diameter of the circumferential groove 132. When inserted into the circumferential groove 132, the top of the annular ring 134 slightly protrudes radially out of the top of the groove. The handle portion 12 preferably has a hollow elongated channel 17 extending between the proximal support 120 and the distal neck 15.

As particularly depicted in FIG. 30, an elongated shaft 22 inserts into the elongated channel 17 of the handle portion 12. The shaft 22 is preferably constructed of rigid material, for example metal. The shaft 22 has a proximal end 130 that includes an insert, for example a pair of opposing parallel flat surfaces cut away from the shaft circumference. The shaft 22 has a distal end that includes a head 36. The head 36 is depicted to have a cylindrical shape with a larger diameter than the shaft diameter. As depicted particularly in FIG. 15, the head 36 has three radial bores 78, 80 (and one not shown) into the outer circumferential surface, preferably used for cleaning. The radial bores 78, 80 (and one not shown) are offset from the central axis of the handle or knob 18 described below, and are offset from each other along a common circumferential line and in a triangular geometry, for example at 120° from each other. The radial bores 78, 80 (and one not shown) improve the cleanliness of this part of the instrument. The head 36 also has a plurality of longitudinal bores 76 extending into the head from the front distal face surface of the head. As depicted, there are three front face longitudinal bores 76, but an alternate number of bores can be provided. Each front face longitudinal bore 76 is arranged in a geometrically-similar positioning with the radial bores 78, 80 (and one not shown), for example at 120° from each other, so that a front face bore intersects with a geometrically-related circumferential bore to form a pathway.

As particularly depicted in FIGS. 26-28, the handle or knob 18 secures over the support 120 and onto the proximal end of the shaft 22. The handle or knob 18 has an inner volume 124 defined by an outer circumferential shell 122. The handle or knob 18 has a central axis extending through the inner volume 124 centrally to the circumferential shell 122. The circumferential shell 122 preferably has a textured circumferential surface and a wall surface 129 that extends from the textured circumferential surface. A receiver 126, for example a bore-shaped receiver, extends into the interior of the wall surface 129 from within the inner volume 124. The receiver 126 has a generally cylindrical shape with a pair of opposing parallel flat planar surfaces 128. The inner volume 124 has a circumferential lip 131 extending inwardly from the open circumference and an inner circumferential surface 133 beyond the lip. The circumferential lip 131 has an inner diameter that is smaller than the diameter of the inner circumferential surface 133. The diameter of the inner surface 133 is slightly larger than the outer diameter of the handle portion support 120. The diameter of the inner circumferential surface 133 is slightly smaller than the diameter of the annular ring 134 in the support 120. The inner diameter of the lip 131 is slightly smaller than the diameter of the annular ring 134.

In use, the proximal end 130 of the shaft 22 is inserted into the distal end of the handle elongated channel 17 and exits out of the proximal end of the handle elongated channel, as depicted in FIGS. 29 and 31. An annular ring 23, preferably constructed of elastic material, secures between the shaft 22 and the proximal end of the handle elongated channel 17. The shaft 22 can rotate with respect to the inner surface of the elongated channel 17 of the handle or knob 18.

The proximal end 130 of the shaft 22 is inserted into the receiver 126 in the handle or knob 18. Preferably, the pair of opposing parallel flat surfaces on the shaft proximal end 130 engage the pair of opposing parallel flat surfaces 128 in the receiver 126 to prevent rotation of the shaft 22 with respect to the receiver 126. The knob inner volume lip 131 snaps over the support annular ring 134. Because the knob inner circumferential surface 133 has a smaller diameter than the annular ring 134 and a larger diameter than the support 120, the handle or knob 18 can rotate with respect to the support 120. When the proximal end of the shaft 22 is secured within the receiver 126, rotation of the handle or knob 18 correspondingly rotates the shaft with respect to the handle elongated channel 17. Alternatively, rotation of the handle or knob 18 can be performed through a powered motor.

The bow arm portion 14 includes a bow 34 with distal end 16 and proximal end 60. The bow arm portion 14 is preferably constructed of durable and rigid material, for example metal. As particularly depicted in FIGS. 4, 11 and 12, the bow arm portion 14 has an arc shape that gradually and smoothly offsets along a vertical axis Z with a gradual incline from the proximal end 60 to the peak, and a stepper decline from the peak to the distal end 16. As depicted for example, the bow arm portion 14 can have a shape that corresponds to a common chicken egg shell 22. As particularly shown in FIGS. 11 and 12, the bow 34 can have an elongated channel 59. Preferably, the channel 59 defines a U-shape with a pair of opposing side-walls 54, 56, a floor 62 extending between the side-walls, and an open top. The side walls 54, 56 are resiliently flexible and naturally return inwardly to a resting position after being temporarily separated. The side walls 54, 56 can include at least one pair of opposing cut-out apertures 58. As depicted, three pairs of opposing elongated receiver seat grooves 46, 48, 50 extend down from the top edges of the interior surface of each opposing side-wall 54, 56. The angle of each groove seat 46, 48, 50 is preferably generally perpendicular to the local axis of the elongated spine 32 described below. The channel floor 62 can have a proximal rear aperture 64, a middle aperture 66 and a distal front aperture 68. As depicted, the most distal groove seat 50 is positioned between the distal aperture 68 and the middle aperture 66.

As particularly depicted in FIGS. 18-22, the bow proximal end 60 has a male insert 38 that can have a hollow cylindrical shape with an open end 106 that provide access to the elongated channel 59. A defined length of the male insert 38 outer surface has a smooth outer surface. A defined length of the male insert 38 outer surface, between the proximal end and the elongated channel 59, has a fastener 52, for example a threaded surface. The male insert 38 has an elongated recession 91 that extends across a predefined length of the smooth outer surface and the treaded surface 52. The male insert 38 has a narrow channel path recession 93 extending from the elongated recession 91 across the remainder of the length of the threaded surface 52. The elongated channel floor 62 has a narrow elongated channel 77 extending between the proximal rear aperture 64 and the narrow channel path recession 93.

As depicted, a wrench 20 is rotatably secured around the male insert threaded surface 52. The depicted wrench 20 has a cylindrical collar shape with a threaded inner surface that corresponds to the male insert threaded surface 52. As shown in FIGS. 23-25, the wrench 20 translates distally and proximally along the R axis when rotated with respect to the nose protrusion threaded surface 52. The wrench 20 has a circumferential receiver recession 112 cut away around the proximal rear inner circumference of the inner surface. The circumferential receiver recession 112 has a continuous smooth surface. A translating ring 114 inserts independently within the wrench receiver recession 112. The translating ring 114 has a cylindrical shape with a smooth inner and outer surface. Rotation of the wrench 20 causing proximal translational movement toward the handle portion 12 engages and forces a corresponding translational movement of the translational ring 114 in the proximal rear direction. Rotation of the wrench 20 causing translational distal movement away from the handle 12 releases the translational ring 114 from the forced rearward movement.

The translating ring 114 has a cut-away aperture 116 and a fastener-receiver aperture 108. As particularly depicted in FIGS. 21-22 and 36, the fastener-receiver aperture 108 aligns with the receiver elongated recession 91. A fastener 110, for example a peg or screw, is secured within the fastener-receiver aperture 108.

As particularly depicted in FIGS. 5, 6, 8, 9 and 10, the spine 32 is secured within the bow elongated channel 59. The spine 32 has a proximal end 72 and a distal end 74. The spine 32 has vertically-offset shape that corresponds to extend within the shape and angles of the bow elongated channel 59. The spine 32 can be constructed of one rod 70, more than one parallel rod 70, or preferably three, parallel rods 70 as particularly depicted in FIGS. 14 and 16. Each rod 70 extends from a position offset from the central axis of the handle or knob 18. Each rod 70 is constructed of durable and rigid material, for example metal. As depicted, each rod has five distinct sections. Each distinct section is rigid and linear. The sections are separated by curved elbows. As depicted particularly in FIG. 14, the proximal 72 and distal 74 ends of each rod 70 have two distinct sections that extend along a common axis A. Between the proximal 72 and distal 74 ends, each rod 70 has a section that extends along an axis B that is parallel to and offset from axis A. Each rod 70 has a pair of sections that extend at oblique angles along axes X and Y between axis A and axis B. Axis X can extend at an incline angle of between 0° to 90° with respect to axis A, more preferably between 10° to 50° incline with respect to axis A, and most preferably about 30° incline with respect to axis A. Axis Y can extend at an angle of between 0° to 90° decline with respect to axis B, more preferably between 20° to 60° decline with respect to axis B, and most preferably about 40° decline with respect to axis B.

The spine 32 can utilize one or more housing cylinders or bundlers 26, 28, 30 to receive and guide the rod or rods 70 in order to maintain the parallel nature of the rods from the proximal and 72 to the distal end 74. Each housing cylinder or bundler 26, 28, 30 can have a generally-circular ring bulge lip insert 40, 42, 44 around the edge of the front distal end circumference. In use within the elongated channel 59, each bulge lip receiver 40, 42, 44 is rotatably received and supported within a receiver seat 46, 48, 58 positioned along the interior surfaces of the side walls 54, 56. The outer diameter of each bulge lip insert 40, 42, 44 is slightly smaller than the distance between each receiver seat 46, 48, 58 so that the lip insert can rotate with respect to the receiver seat. When the bulge lip receivers 40, 42, 44 are inserted within the receiver seat 46, 48, 50 the spine 32 can rotate within the receiver seats with respect to the elongated channel 59. The bulge lip receivers 40, 42, 44 can alternatively include ball bearings (not shown) to improve rotation within the receiver seats 46, 48, 50.

As particularly depicted in FIG. 15, the example housing cylinder or bundler 28 can be a unitary in construction and have a generally-cylindrical structure with at least one, preferably three, elongated semi-circumferential channels cut out along the length of the outer circumference and extending from the proximal end of the bundle to the distal end. As depicted, the channels can be arranged in triangular geometry with respect to each other, for example separated by 120° with respect to each other. The rods 70 can be snapped into the respective semi-circular guide channel. The rods 70 can be removed from the guide 26, 28, 30 channels to be cleaned. When the rods 70 are snapped into the channels, each rod can freely rotate with respect to its particular channel circumference, and thus rotate with respect to the bundle guide.

The example angles of section axes A, B, X, Y, described above, are such that, except for the bulge lips 40, 42, 44, the spine 32 can rotate freely within the relevant opposing receiver seat 46, 48, 50 of the elongated channel 59 without contacting the inner surfaces of the bow 34. During rotation, each axial section of the spine 32 rotates, or more specifically shifts position with respect to the other rods and bow 14, in parallel to the relevant axis, and distinct from the other axes.

As particularly depicted in FIGS. 9, 15 and 16, the proximal end 72 of the spine 32 removably and rotatably secures within the front face of the shaft head 36. The proximal end 72 of each rod 70 removably and rotatably inserts within a respective head front face bore 76 extending within the head 36 from the front face. Each rod 70 is removably and rotatably inserted into a bore 76 until it spans the interior opening of a corresponding radial surface bore 78, 80 (and one not shown). As depicted, the rods 70 are positioned within the bores 76 in a parallel arrangement 120° with respect to each other. The rods 70 can be removed from the shaft head 36 for cleaning.

During rotation of the handle or knob 18, the shaft head 36 is correspondingly rotated, causing the shaft head bores 76 to rotatably transition position with respect to each other, corresponding to the head rotation. When the shaft head bores 76 are rotated, the rods 70 are rotatably repositioned with respect to each other, corresponding to the rotation of the head 36. Because each rod 70 is rotatably secured within the shaft head bore 76, and each bundle guide lip 40, 42, 44 is rotatably secured within a corresponding seat receiver 46, 48, 50, each rod axis A, B, X and Y, maintains its orientation within the bow 14 as the rods are being repositioned with respect to each other. Similarly, because each rod 70 is rotatably secured within an elongated channel of the bundle guide 26, 28, 30, and each bundle guide lip 40, 42, 44 is rotatably secured within a corresponding seat receiver 46, 48, 50, each rod axis A, B, X and Y, maintains its orientation within the bow 14 as the rods are being repositioned with respect to each other. Similarly, if the spine 32 includes a single rod 70, the rod maintains its axial orientation (A, B, X, Y) with respect to the bow 14 as the shaft head 36 and bundle guides 26, 28, 30 are rotated.

As particularly depicted in FIG. 38, the distal end 16 of the bow 34 has a cap 102 with a central aperture 170. The cap 102 secures to the end of the distal end 16, for example with a snap-fit, friction fit or adhesive. Near the distal end 16, a pair of directly-opposing apertures 172 are positioned in vertical alignment with the most-distal floor aperture 68. A pair of fasteners 100, for example screws or pegs, insert through the directly-opposing apertures 172. The fasteners 100 secure within the apertures 172 with a friction fit or adhesive. The pair of fasteners 100 are secured through each length of the aligned apertures 172, 146, 148. When secured with the fasteners 100, the swivel receiver is supported between the side-walls 54, 56, but can rotate or pivot back and forth, as particularly depicted in FIG. 32.

As particularly depicted in FIGS. 33-38, a swivel receiver 84 is inserted through the distal-most floor aperture 68. The swivel receiver 84 has a longitudinal aperture 144 and a pair of opposing horizontal apertures 146, 148. The horizontal apertures have a diameter substantially similar to the side-wall apertures 172. When the swivel receiver 84 is inserted between the side walls 54, 56 within the elongated channel 59, apertures 146, 148 align with apertures 172, and the pair of fasteners 100 are secured through the side-wall apertures 172 and then through a corresponding swivel receiver horizontal aperture 146, 148.

As particularly depicted in FIGS. 40-42, the prehension fastener 190 is inserted through the cap central aperture 170 and the swivel receiver longitudinal aperture 144. The depicted prehension fastener 190 has a receiver body 194 with a cylindrical shape. The prehension fastener 190 has a circumferential stopper lip 202 and a neck 204 to support a prehension grip 192. The prehension grip 192 can be a threaded surface, for example a screw. The prehension fastener 190 has a unitary construction that is durable and rigid, for example with metal.

The prehension fastener receiver body 194 has a plurality of longitudinal bores 196 extending from a proximal rear face into the receiver body. Preferably, the receiver body 194 has an equivalent number of longitudinal bores 196 as the number of rods 70 in the spine 32, and the bores are oriented with respect to each other similarly to the orientation of the rods, for example 120° with respect to each other. In use, the distal ends of the spine rods 70 are removably and rotatably inserted into the receiver body longitudinal bores 196.

When the spine rods 70 are removably and rotatably inserted into the prehension fastener receiver bores 196, the previously described rotation of the handle or knob 18 and shaft head 36 causes a corresponding rotation of the prehension fastener 190. The rotation of the prehension fastened 190 causes a corresponding rotatable respositioning of the reprehension fastener receiver bores 196 with respect to each other. Because each rod 70 is rotatably secured within a prehension fastener receiver bore 196, each rod axis A, B, X and Y, maintains its orientation within the bow 14 as the rods are being repositioned with respect to each other.

The rods 70 can be removed from the prehension fastener longitudinal bores 196 for cleaning. The prehension receiver body 194 preferably has a pair of directly opposing radial surface bores 198, 200 that extend into the receiver body from the circumference to allow for cleaning.

FIG. 42 depicts an enclosure pipe collar 206 that can be fitted over the prehension fastener grip 192 and neck 204. The enclosure pipe collar 206 has a base lip 210 and a cylindrical body 208. When fitted over the prehension fastener 190, the base lip 210 engages the prehension stopper lip 202. A closing ring 212 preferably fits over the prehension grip 192 and fits within the cap central aperture 170 with a friction fit. A commercially available acetabular cup can be gripped on the prehension grip 192 during use.

As particularly depicted in FIGS. 33-35, the swivel receiver body 194 has a cut-away notch 150. Preferably, the cut-away notch 150 is located at a lower corner of the receiver body 194. The cut-away notch 150 preferably has a horizontal surface and a vertical surface. The horizontal surface has a pair of vertically-oriented bores or apertures 152, 154. Preferably, the pair of bores or apertures 152, 154 have a conical or frustoconical shape. The vertical surface can have a pair of split semi-circular recessed cutouts 156, 158. A pair of fasteners 140, 142, for example plugs or pins, secure within the pair of bores or apertures 152, 154. The pair of fasteners 140, 142 can be secured within the pair of bores or apertures 152, 154 with adhesive, welding or a friction fit. Preferably, each of the pair of fasteners 140, 142 have a conical or frustoconical pin shape to correspond with the pair of bores or apertures 152, 154 and fit within the recessed cutouts 156, 158. In use, a cable or cord 82 is pinch-secured between the pair of fasteners 140, 142 and the cut-away vertical surface, specifically the split semi-circular recessed surfaces 156, 158. The cable or cord 82 can be constructed of durable and rigidly-flexible material, for example metal wire or plastic. As depicted in FIG. 38, the receiver body 194 is positioned or oriented so that the cable or cord 82 and fasteners 140, 142 are engaged near, or substantially proximal to, the elongated channel floor 62.

As particularly depicted in FIGS. 8, 32 and 37, the bow 34 can have several guide pins 88, 90, 92, 94, 96 and 98 that are secured, and preferably extend, between the opposing side-walls 54, 56. The series of guide pins 88, 90, 92, 94, 96 and 98 guide the path of the cable or cord 82 within the elongated channel 59. The cable or cord 82 can be guided over the top of the middle guide pins 92, 94 or alternatively can be guided under the bottom of the end guide pins 88, 90, 96, 98. The guide pins 88, 90, 92, 94, 96 and 98 can have a circumferential channel that receives and maintains the position of cable or cord 82.

In use, as particularly depicted in FIGS. 21 - 22, the cable or cord 82 extends through and/or along the narrow elongated floor channel 77 and along the narrow receiver channel recession 93 and into the receiver elongated recession 91. The cable or cord 82 is aligned with the fastener receiver 108 and the fastener 110 tightens into the fastener receiver to apply a pinching pressure force onto the cord or cable 82 against the receiver elongated recession 91.

In use, the cable or cord 82 is secured at either end by, and extends between, the swivel receiver fasteners 140, 142 and the translating ring fastener 110, described above. When the wrench 20 is rotated to cause rearward translational movement of the wrench and the translational ring 114, the cable or cord 82 is pulled proximally rearward. When the cable or cord 82 is pulled rearward, the swivel receiver 84 is pivoted about the pair of fasteners 100 such that the bottom surface of the swivel receiver is pulled rearward. The rearward pivot movement causes the bottom distal front edge of the swivel receiver 84 to apply pressure to the outer surface of the prehension fastener receiver body 194. When this pressure is applied by the swivel receiver 84, the prehension fastener 190 is prevented from rotating. Correspondingly, when the wrench 20 is rotated to cause forward translational movement, the tension on the cable or cord 82 is relieved and the swivel receiver 84 can pivot forward to a relaxed state, allowing the prehension fastener 190 to rotate.

It is to be understood that this invention is not limited to the specific devices, methods, conditions, or parameters described and/or shown herein, and that the terminology used herein is for the purpose of describing particular embodiments by way of example only. Thus, the terminology is intended to be broadly construed and is not intended to be limiting of the claimed invention. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the invention has been shown and described in exemplary forms, it will be apparent to those skilled in the art that many modifications, additions, and deletions can be made therein without departing from the spirit and scope of the invention as defined by the following claims.

What is claimed is:

1. An apparatus for surgery, the apparatus comprising:
an elongated bow arm comprising a substantially arced shape, the elongated bow comprising an elongated channel extending between a proximal end and a distal end;
a handle comprising a central axis and being rotatably secured with respect to the elongated bow arm proximal end;
a spine received within the elongated bow arm channel and comprising at least one rigid rod comprising a distal end and a proximal end and having a shape that substantially follows the substantially arced elongated bow arm, the spine being offset from the handle axis and being rotatably secured with respect to the handle.

2. The apparatus of claim 1, comprising at least two parallel rigid rods, the at least two rigid rods comprise a plurality of linear sections extending along distinct axes.

3. The apparatus of claim 2, wherein the at least two parallel rigid rods comprise a proximal section and a distal section extending along a common linear axis.

4. The apparatus of claim 3, wherein the at least two parallel rigid rods comprise: an incline section extending from the proximal section along an axis oblique to the proximal section axis, and a decline section extending from the distal section along an axis oblique to the distal section.

5. The apparatus of claim 4, wherein the at least two parallel rigid rods comprise a connector section extending between the incline section and the decline section, the connector section extending along an axis parallel to and offset from the proximal section and distal section.

6. The apparatus of claim 5, wherein the connector section axis is oblique to the incline axis and oblique to the decline axis.

7. The apparatus of claim 5, further comprising:
a first bundle housing to rotatably guide the rigid rod incline sections in parallel with respect to each other; and
a second bundle housing to rotatably guide the rigid rod decline sections in parallel with respect to each other.

8. The apparatus of claim 7, further comprising:
a third bundle housing to rotatably guide the rigid rod connector sections in parallel with respect to each other.

9. The apparatus of claim 5, wherein the first bundle guide comprises a substantially-circumferential lip, the second bundle receiver comprises a substantially-circumferential lip, and the bow arm comprises a pair of seats to rotatably receive the first bundle housing substantially-circumferential lip and the second bundle housing substantially-circumferential lip.

10. The apparatus of claim 9, wherein a first bow arm seat comprises an elongated groove extending along an axis normal to the incline section axis, and a second bow arm seat groove comprises an elongated groove extending along an axis normal to the decline section axis.

11. The apparatus of claim 1, further comprising a prehension fastener rotatably secured to the spine distal end.

12. An apparatus for surgery, the apparatus comprising:
a spine comprising at least one rigid rod comprising:
a proximal section and a distal section separated by a horizontal axis,
a linear incline section extending from the proximal section along an axis oblique to the horizontal axis, and
a linear decline section extending toward the distal section along an axis oblique to the horizontal axis;
a rotatable handle comprising a central axis and being rotatably secured with respect to the spine proximal end, the at least one rigid rod being offset from the rotatable handle central axis.

13. The apparatus of claim 12, comprising at least two parallel rigid rods, the at least two parallel rigid rods further comprise a linear connector section extending between the linear incline section and the linear decline section.

14. The apparatus of claim 13, wherein the linear connector section extends along an axis parallel and offset to the horizontal axis.

15. The apparatus of claim 12, further comprising an elongated bow arm comprising a substantially arced shape, the elongated bow comprising an elongated channel to receive the spine.

16. The apparatus of claim 15, wherein the rotatable handle rotatably secures to a proximal end of the elongated bow arm.

17. The apparatus of claim 15, wherein the elongated bow arm channel comprises at least two seat receivers, and the spine comprises at least two circumferential bundlers comprising a lip, the at least two circumferential bundler lips being rotatably received within the at least two seat receivers.

18. The apparatus of claim 17, wherein a first circumferential bundler is rotatably secured to the incline section of the at least two rigid rods, and a second circumferential bundler is rotatably secured to the decline section of the at least two rigid rods.

19. The apparatus of claim 18, wherein a first seat receiver comprises an elongated groove extending along an axis normal to the incline section axis, and a second seat receiver comprises an elongated groove extending along an axis normal to the decline section axis.

20. A method for performing surgery, the method comprising:
removably securing an medical insert to a prehension fastener, the prehension fastener being removably secured to the distal end of a spine received within a bow arm comprising a distal end and a proximal end, the spine comprising at least one rigid rod comprising:
a proximal section and a distal section separated by a horizontal axis,
a linear incline section extending from the proximal section along an axis oblique to the horizontal axis, and
a linear decline section extending toward the distal section along an axis oblique to the horizontal axis;
rotatably securing a rotatable handle to the proximal end of the bow arm; and
controlling the rotation of the prehension fastener by rotating the rotatable handle.
